(19) United States
(12) Patent Application Publication
(10) Pub. No.: US 2020/0369587 A1
(43) Pub. Date: Nov. 26, 2020

(54) PROCESS FOR THE HYDROXYLATION OF AN AROMATIC COMPOUND

(71) Applicant: RHODIA OPERATIONS, Aubervilliers (FR)

(72) Inventor: Laurent GAREL, Lyon (FR)

(21) Appl. No.: 16/771,101

(22) PCT Filed: Dec. 14, 2018
(86) PCT No.: PCT/EP2018/084932

§ 371 Date: Jun. 9, 2020

(30) Foreign Application Priority Data
Dec. 15, 2017 (FR) 1762235

(51) Int. Cl. C07C 41/26; C07C 41/40

(52) U.S. Cl. CPC C07C 41/26; C07C 43/23; C07C 41/40

(57) ABSTRACT

The present invention relates to a process for the hydroxylation of an aromatic compound comprising at least one alkoxy group, comprising a step (a) of reacting said aromatic compound comprising at least one alkoxy group with hydrogen peroxide in the presence of a catalyst, in a solvent comprising water, an alcohol, or a mixture of alcohols.

The present invention relates to a process for the hydroxylation of an aromatic compound comprising at least one alkoxy group by reaction of said aromatic compound with hydrogen peroxide in the presence of a catalyst.

Hydroxylated aromatic compounds are important in the field of organic synthesis. Different routes for the synthesis of these products have been developed over time, in particular by hydroxylation of phenol in the presence of a catalyst. For example, the reaction for the hydroxylation of phenol results in two isomers being obtained, namely 1,4-dihydroxybenzene or hydroquinone (HQ) and 1,2-dihydroxybenzene or catechol (PC), which are compounds having a high industrial potential. These hydroxylated aromatic compounds are used in numerous fields of application, such as polymerization inhibitors, pharmaceutical agents, agrochemical agents, perfumery or the food industry.

Given this broad field of operation, it is necessary to manufacture these products on an industrial scale and to have optimized manufacturing processes.

Conventionally, dihydroxy aromatic compounds are produced by hydroxylation of phenol with hydrogen peroxide in the presence of an acid catalyst which is a strong protic acid (see FR 2 071 464) or in the presence of a solid catalyst having acidic properties such as, for example, a solid catalyst having acidic properties such as, for example, a TS-1 zeolite (FR 2 489 816), or an MEL titanosilicalite zeolite (EP 1 131 264), an MFI titanosilicalite zeolite (EP 1 123 159) or an MCM-22 zeolite.

The hydroxylation of aromatic compounds is also described in the following documents: J. Chem. Soc. Chem. Commun. 1995, 349-350, Applied Catalysis A: General 327 (2007) 295-299, Microporous and Mesoporous Materials 21 (1998) 497-504, Catalysis Today 49 (1999) 185-191, Ind. Eng. Chem. Res. 2007, 46, 8657-8664, J. Mater. Chem. 2000, 10, 1365-1370, U.S. Pat. No. 5,426,244, EP 0 919 531, FR 2 489 816, EP 0 200 260, Catal. Sci. Technol. 2015, 5, 2602-2611, Tetrahedron Lett. 1983, 24(44), 4847-4850, J. Am. Chem. Soc. 1988, 110, 7472-7478, Bull. Chem. Soc. 1989, 62, 148-152, Chem. Sci. 2017, 8, 8373-8383, Adv. Synth. Catal. 2015, 357, 2017-2021, Journ. Mol. Catal. A: Chemical, 2015, 408, 262-270.

One of the difficulties of these processes is generally that of optimizing the productivity of the reaction in order to meet the demand for said hydroxylated aromatic compounds. The optimized parameters may include reaction yields, the ratio of hydroxylated aromatic isomers, or the energy efficiency of the reaction.

In order to respond to this general productivity problem, numerous documents mention specific reaction conditions. For example, the nature of the solvent or solvents used for the reaction is described in the scientific publication by Thangaraj et al., Indian Journal of Chemistry, vol. 33A, March 1994, p. 255-258.

Under these circumstances, the present invention solves the problem of providing a process for producing a hydroxylated aromatic compound comprising at least one alkoxy group, preferably for producing a monohydroxylated aromatic compound, the process being highly selective for one isomer with respect to the other isomer, while limiting the amount of by-products formed and maintaining a high yield and high productivity. The reaction of the present invention may also be adjusted to select the major isomer. Indeed, depending on the end use of the hydroxylated aromatic compound, only one isomer may be required. For example, for the synthesis of guaifenesin, of vanillin or of ethylvanillin, guaiacol or guethol, which are the respective ortho-hydroxylated products of anisole and phenetol, would be necessary, while for polymerization inhibitor functionalities, para-methoxyphenol, which is a product of anisole para-hydroxylation, would be used.

The present invention relates to a process for the hydroxylation of an aromatic compound comprising at least one alkoxy group, comprising a step (a) of reacting said aromatic compound comprising at least one alkoxy group with hydrogen peroxide in the presence of a catalyst, in a solvent comprising water, an alcohol, or a mixture of alcohols.

Another subject of the present invention relates to a hydroxylated aromatic compound comprising at least one alkoxy group obtainable by the process of the present invention.

In the present description, and unless otherwise indicated, the expression “between . . . and . . . ” includes the limits.

In the present description, and unless otherwise indicated, the expression “alkyl” denotes a linear or branched, saturated or unsaturated hydrocarbon-based chain comprising from 1 to 6 carbon atoms.

In the present description, and unless otherwise indicated, the expression “alkoxy” represents an alkyl group bonded to an oxygen atom: R—O.

A first aspect of the present invention relates to a process for the hydroxylation of an aromatic compound comprising at least one alkoxy group, comprising step (a) of reacting said aromatic compound comprising at least one alkoxy group with hydrogen peroxide in the presence of a catalyst, in a solvent comprising water, an alcohol or a mixture of alcohols.

Step (a) is a reaction for hydroxylation of an aromatic compound comprising at least one alkoxy group. Step (a) typically results in the formation of hydroxylated aromatic compounds in the form of isomers. Advantageously, the process according to the present invention makes it possible to predict the ratio between the isomers.

The aromatic compound comprising at least one alkoxy group according to the present invention is a compound of formula (I) wherein R is a linear or branched, saturated or unsaturated alkyl group comprising from 1 to 6 carbon atoms; preferably, R is chosen from the group consisting of methyl, ethyl, isopropyl, butyl and tert-butyl; preferably, the group R is chosen from the group consisting of methyl or ethyl.

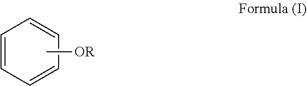

In a preferred aspect of the present invention, the compound of formula (I) is substituted with 1 or 2 alkoxy groups, in a preferred aspect, the compound of formula (I) is substituted with 1 alkoxy group. Consequently, in a preferred aspect of the present invention, the compound of formula (I) is chosen from the group consisting of anisole or phenetol.

Optionally, the compound of formula (I) may be substituted with other groups, for example the substituted aromatic compound comprising at least one alkoxy group may also comprise an alkyl group optionally substituted with heteroatoms. For example, the compound of formula (I) may be substituted one, two, three or four times with a group chosen from methyl, ethyl, propyl and butyl.

When the compound of formula (I) is anisole or phenetol, the reactions and products are described in scheme 1:

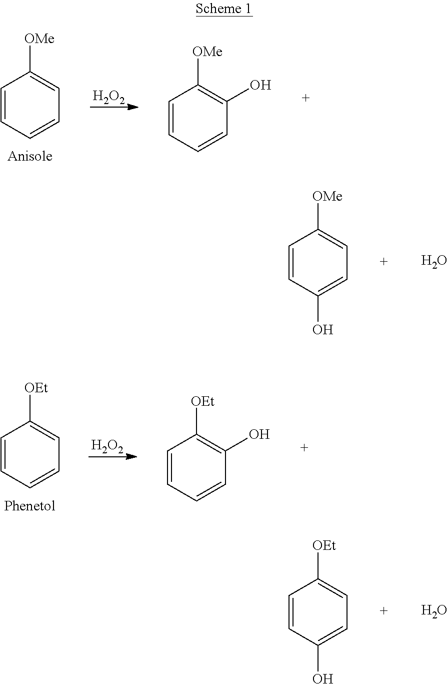

The hydroxylation reaction (step (a)) makes it possible, in the case of anisole, to produce a mixture of guaiacol (GA) and para-methoxyphenol (PMP), and in the case of phenetol, to produce a mixture of guetol (GE) and para-ethoxyphenol (PEP). More generally, the hydroxylation reaction allows the production of a mixture of ortho-alkoxyphenol and para-alkoxyphenol. Advantageously, the process according to the present invention makes it possible to select the desired ortho/para ratio. Preferably, the ortho/para ratio is less than 1, more preferably less than or equal to 0.7, even more preferably less than or equal to 0.4, and most preferably less than or equal to 0.2. In one preferred embodiment, the GA/PMP molar ratio is less than 1, more preferably less than or equal to 0.7, even more preferably less than or equal to 0.4, and most preferably less than or equal to 0.2. In one preferred embodiment, the GE/PEP molar ratio is less than 1, more preferably less than or equal to 0.7, even more preferably less than or equal to 0.4, and most preferably less than or equal to 0.2.

The present invention may be carried out by any of a batch process, a semi-batch process and a continuous-flow process. Various types of reactor may be used to carry out the process according to the invention. Advantageously, the process according to the invention is carried out in a stirred reactor or a cascade of stirred reactors or, as a variant, in a plug-flow reactor, for example a tubular reactor which is placed horizontally, vertically or inclined. Preferably, the catalyst of the present invention is a heterogeneous catalyst, preferably a zeolite comprising titanium and, more preferably, a titanosilicate zeolite, preferably chosen from the group consisting of MFI, MEL, TS-1, TS-2, Ti-MWW, Ti-MCM68, and even more preferably TS-1. Preferably, the zeolite has a Ti/(Ti+Si) molar ratio of from 0.0001 to 0.10 and preferably from 0.0001 to 0.05, for example from 0.005 to 0.04. Titanosilicalite may be prepared by any publicly known process. Although the titanosilicate catalyst may be used as it is, it may be used after having been molded. As process for molding the catalyst, extrusion molding, tablet making, tumbling granulation, spray granulation or the like is generally used. When the catalyst is used in the fixed bed process, extrusion molding or tablet making is preferable. In the case of the suspension bed process, spray granulation is preferable and, as described in, for example, U.S. Pat. No. 4,701,428, a process comprising mixing a suspension of titanosilicate prepared in advance with a binder, for example silica or alumina, and carrying out spray granulation using a spray dryer is a general process.

Advantageously, if the reaction is carried out in concentrated suspension, the amount of titanosilicate catalyst used is preferably in the range of 0.1 to 30% by mass, more preferably 0.5 to 20% by mass, and most preferably 1 to 20% by mass in terms of external ratio on the basis of the total mass of the reaction medium. When the amount of the catalyst is not less than 0.1% by mass, preferably not less than 0.5% by mass, more preferably not less than 1% by mass, the reaction is complete in a short time and the productivity is increased, so that such an amount is preferable. When the amount thereof is not more than 30% by mass, preferably not more than 20% by mass, the amount of the catalyst to be separated and recovered is small, so that such an amount is preferable.

Preferably, the oxidizing agent is used in a molar ratio, relative to the aromatic compound comprising at least one alkoxy group, of from 0.005 to 0.60, preferably from 0.05 to 0.50 and even more preferably from 0.15 to 0.35. Although the concentration of hydrogen peroxide used is not specifically restricted, a usual aqueous solution having a concentration of 30% may be used, or an aqueous solution of hydrogen peroxide with a higher concentration may be used as it is or may be used after having been diluted with a solvent which is inert in the reaction system. Examples of the solvent used for dilution include an alcohol, preferably chosen from the group consisting of methanol, ethanol, isopropanol, n-butanol or tert-butanol and water. Depending on the choice of reaction mode, the hydrogen peroxide may be added all at once or may be added gradually over a long period of time.

Advantageously, the process of the present invention is carried out in a solvent comprising water, an alcohol or a mixture of alcohols. Preferably, the alcohol is chosen from alcohols having 1 to 6 carbon atoms, preferably alcohols comprising a tertiary or quaternary carbon atom. Examples of alcohols comprising a tertiary or quaternary carbon atom include tert-butanol, 2-methyl-1-propanol, 2-methyl-1-butanol, 3-methyl-1-butanol, 2,2-dimethylpropanol, 2-methyl-2-butanol and 3-methyl-2-butanol. Among these, tert-butanol, 2,2-dimethyl-1-propanol and isopropanol are preferable. The solvent may be used alone or in the presence of a co-solvent. The co-solvent may be chosen from water, acetone, acetonitrile, 1,4-dioxane or another alcohol, preferably chosen from the group consisting of methanol, ethanol, isopropanol, n-propanol, n-butanol or tert-butanol. Advantageously, the mass ratio between the solvent and the co-solvent used in the reaction is between 1:99 and 99:1, preferably between 10:90 and 90:10. The amount of the alcohol or mixture of alcohols used is preferably in the range of 1 to 90% by mass, more preferably 3 to 50% by mass, on the basis of the total mass of the reaction liquid.

The amount of water used in the present invention may be the water contained in the aqueous solution of hydrogen peroxide. The amount of water is preferably in the range of 5 to 90% by mass, more preferably in the range of 8 to 90% by mass, even more preferably in the range of 8 to 85% by mass on the basis of the total mass of the reaction liquid.

The reaction temperature may be greater than or equal to 30° C., preferably greater than or equal to 40° C. The reaction temperature may be less than or equal to 130° C., preferably less than or equal to 100° C. The reaction may be carried out at atmospheric pressure. The reaction may be carried out at a pressure of 10 bar or less, preferably less than or equal to 6 bar.

The present reaction may be carried out batchwise, or may be carried out semi-batchwise, or may be carried out continuously, for example in a plug-flow reactor model of the fixed bed flow type. In addition, a plurality of reactors may be connected in series and/or in parallel. The number of reactors is preferably from 1 to 4 depending on the cost of equipment. When a plurality of reactors are used, the hydrogen peroxide may be placed therein in a divided manner.

When the present reaction is carried out in a concentrated suspension mode, a step of separating the catalyst from the reaction liquid is preferably included. For the separation of the catalyst, precipitation separation, a centrifugal filter, a vacuum belt filter, a pressure filter, a filter press, a fabric filter, a rotary filter or the like is used, whether in the horizontal or vertical configuration. In the case of a continuous filter such as a rotary filter, a concentrated suspension of catalyst, which is that obtained after a liquid phase has been withdrawn from the reaction liquid containing the catalyst, may be used for the reaction again. When the reaction is carried out continuously, the liquid phase is withdrawn continuously. When the catalyst is separated not in the form of a suspension, but in the form of a cake, it may be used for the reaction again as it is, or it may be used for the reaction again after having been subjected to a regeneration treatment. The regeneration treatment comprises multiple steps of washing the catalyst, inerting, evaporation of the solvent, controlled organic deposit oxidation. For the regeneration treatment, a tray dryer, a belt dryer, a rotary dryer, a spray dryer, an instant dryer or the like is used. The regeneration treatment may be carried out in an atmosphere of an inert gas such as nitrogen, an air atmosphere, an atmosphere of air diluted with an inert gas, the amount of oxygen during the regeneration treatment is preferably controlled, the amount of oxygen is generally less than 10%, preferably less than 8%, most preferably less than 5%, a water vapor atmosphere, an atmosphere of water vapor diluted with an inert gas, or the like. The drying temperature is preferably from 60 to 800° C., particularly preferably from 100 to 700° C., most preferably from 150° C. to 650° C. When the regeneration temperature is this temperature, the organic substances that have adhered may be removed without significant degradation of the catalyst performance results. The regeneration treatment may also be carried out by combining a plurality of different temperature regions. The regeneration may be carried out at a given frequency on the total catalytic charge or only on a portion thereof after separation of the filtration medium. The portion may be in the range of from 1 to 50% of the catalytic charge, preferably from 2 to 40%, more preferably from 5 to 20% of the catalytic charge. In order to compensate for the irreversible deactivation of the catalyst over time, fresh catalyst may be introduced to maintain the performance results. The amount of fresh catalyst is in the range of from 0 to 20%, as expressed as a function of the total amount of catalyst, more preferably from 0.2 to 10%, even more preferably from 0.5 to 2%.

In order to obtain a hydroxylated aromatic compound comprising at least one alkoxy group from the reaction liquid, a purification treatment such as the separation of the unreacted components and the by-products may be carried out on the reaction liquid or a separated liquid containing a hydroxylated aromatic compound comprising at least one alkoxy group, said separated liquid being the liquid after separation of the catalyst. The process according to the present invention may also comprise a step (b) of purifying the composition obtained after step (a). The purification treatment may be carried out more preferably on the separated liquid containing a hydroxylated aromatic compound comprising at least one alkoxy group, said separated liquid being the liquid after separation of the catalyst. The process for the purification treatment is not specifically limited, and specific examples of the processes include decanting, extraction, distillation, crystallization and combinations of these processes. The process and the procedure of the purification treatment are not specifically limited, but for example, the following process makes it possible to purify the reaction liquid and the separated liquid containing a hydroxylated aromatic compound comprising at least one alkoxy group, said separated liquid being obtained after separation of the catalyst.

The process according to the present invention may also comprise a step (c) of shaping the composition obtained after step (a) or (b) in the form of an amorphous or crystallized powder, of balls, of beads, of pellets, of granules or of flakes.

Another subject of the present invention relates to a hydroxylated aromatic compound comprising at least one alkoxy group obtained by the process of the present invention. The hydroxylated aromatic compound obtained by the process of the present invention contains certain impurities which are derived from the process described in the present invention and, in particular, derived from the use of a specific solvent.

1. A process for the hydroxylation of an aromatic compound comprising at least one alkoxy group, comprising:
(a) reacting said aromatic compound comprising at least one alkoxy group with hydrogen peroxide in the presence of a catalyst, in a solvent comprising water, an alcohol or a mixture of alcohols, wherein:
the catalyst is a zeolite comprising titanium, and
the alcohol is chosen from alcohols having 1 to 6 carbon atoms and comprises a tertiary or quaternary carbon atom.

2. The process as claimed in claim 1, wherein the zeolite is selected from the group consisting of MFI, MEL, TS-1, TS-2, Ti-MWW, and Ti-MCM68.

3. The process as claimed in claim 1, wherein the alcohol is selected from the group consisting of isopropanol, 2,2-dimethylpropanol, and tert-butanol.

4. The process as claimed in claim 1, wherein the solvent comprises a co-solvent selected from the group consisting of water, acetone, acetonitrile, 1,4-dioxane, and other alcohols.

5. The process as claimed in claim 1, further comprising:
(b) purifying the composition obtained after step (a).

6. The process as claimed in claim 1, further comprising:
(c) shaping the composition obtained after step (a) or (b) in the form of an amorphous or crystallized powder, of balls, of beads, of pellets, of granules, or of flakes.

7. A hydroxylated aromatic compound comprising at least one alkoxy group obtained by the process defined in claim 1.

8. The process as claimed in claim 2, wherein the zeolite is TS-1.

9. The process as claimed in claim 4, wherein the solvent comprises a co-solvent selected from water, methanol, ethanol, isopropanol, n-propanol, n-butanol, and tert-butanol.
